(19) United States
(12) Patent Application Publication
(10) Pub. No.: US 2012/0093878 A1
(43) Pub. Date: Apr. 19, 2012

(54) PHARMACEUTICAL COMPOSITIONS CONTAINING A BIGUANIDE AND A THIAZOLIDINEDIONE

(75) Inventors: Romi Barat SINGH, Faridabad (IN); Kalaiselvan RAMARAJU, Tamil Nadu (IN); Sandeep Kumar VATS, Sonipat City (IN); Balaram MONDAL, East Midnapore (IN)

(73) Assignee: RANBAXY LABORATORIES LIMITED, New Delhi (IN)

(21) Appl. No.: 13/193,705

(22) Filed: Jul. 29, 2011

(30) Foreign Application Priority Data
Jul. 30, 2010 (IN) 1811/DEL/2010
Mar. 25, 2011 (IN) 842/DEL/2011

(51) Int. Cl. A61K 9/00; B05D 7/00; A61P 3/10; B05D 5/00; A61K 31/426; A61K 31/4439

(52) U.S. Cl. 424/400; 514/369; 514/342; 427/2.21

(57) ABSTRACT

The present invention relates to pharmaceutical compositions that include a combination of a biguanide present in an extended-release form and a thiazolidinedione present in an immediate-release form. The present invention further relates to the processes for preparing such compositions.

### FIELD OF THE INVENTION

The present invention relates to pharmaceutical compositions that include a combination of a biguanide present in an extended-release form and a thiazolidinedione present in an immediate-release form. The present invention further relates to the processes for preparing such compositions.

### BACKGROUND OF THE INVENTION

Diabetes mellitus is a metabolic disorder characterized by hyperglycemia and insulin resistance, and is often associated with other conditions such as obesity, hypertension, hyperlipidemia, cardiovascular disease, retinopathy, neuropathy, and nephropathy. The disease is progressive in nature but often can be controlled initially by diet alone, although over time it usually requires treatment with drugs, such as sulfonylureas, and/or injections of exogenous insulin. Two major forms of diabetes mellitus are now recognized: Type I and Type II. Type I diabetes, or insulin-dependent diabetes, is the result of an absolute deficiency of insulin, the hormone that regulates glucose utilization; patients with Type I diabetes are dependent on exogenous insulin for survival. Type II diabetes, or non-insulin-dependent diabetes (NIDDM), often occurs concurrent with normal, or even elevated levels of insulin, and appears to be the result of the inability of tissues to respond appropriately to insulin (i.e., insulin resistance). Insulin resistance is a major susceptibility trait of NIDDM and also is a contributing factor in arteriosclerosis, hypertension, lipid disorders and polycystic ovarian syndrome.

Biguanides have been the most widely used class of antidiabetics. They act by increasing insulin activity in peripheral tissues, reducing hepatic glucose output due to inhibition of gluconeogenesis, and reducing the absorption of glucose from the intestine. Metformin, phenformin, buformin, etc., belong to this group. Metformin has been widely prescribed for lowering blood glucose in patients with NIDDM and is marketed in 500, 750, 850 and 1000 mg strengths. However, because it is a short acting drug, metformin requires twice-daily or three-times-daily dosing (500-850 mg tab 2-3/day or 1000 mg bid with meals). Adverse events associated with metformin include anorexia, nausea, vomiting and diarrhea. The adverse events may be partially avoided by taking an extended-release dosage form rather than multiple daily doses. Besides reducing the adverse events, administering an extended-release dosage form provides a reduction in the frequency of administration.

Thiazolidinediones substantially increase insulin sensitivity in muscle, liver, and adipose tissue in several NIDDM animal models, resulting in the correction of elevated plasma levels of glucose, triglycerides and nonesterified fatty acids without the occurrence of hypoglycemia. These agents, (e.g., troglitazone, rosiglitazone and pioglitazone), function by increasing the sensitivity of peripheral tissues, such as skeletal muscle, towards insulin. Pioglitazone, the most widely used thiazolidinedione, is normally administered at doses from about 15 mg to about 45 mg, and is given as a single dose once per day. Another glitazone, rosiglitazone, is administered at doses of about 5 mg to about 10 mg per day.

Insulin resistance is a common feature characterizing the pathogenesis of Type II diabetes. Metformin improves glucose tolerance but cannot enhance insulin sensitivity. In contrast, glitazones improve glycemic control by improving insulin sensitivity. The glitazones are highly selective and potent agonists for the peroxisome proliferator-activated receptor-gamma (PPAR-gamma). Activation of PPAR-gamma nuclear receptors regulates the transcription of insulin responsive genes involved in the control of glucose production, transport, and utilization. In addition, PPAR-gamma-responsive genes also participate in the regulation of fatty acid metabolism. The antidiabetic activity of glitazones has been demonstrated in those Type II diabetes in which hyperglycemia and/or impaired glucose tolerance is a consequence of insulin resistance in target tissues. A single administration of glitazones activates the insulin receptors for an extended period and may thus be administered as a single dose without there being a need to maintain the plasma concentration.

A combination therapy of a biguanide and a thiazolidinedione, therefore, has a synergistic effect on glucose control because both agents act by different but complementary mechanisms.

Formulations comprising biguanides in extended-release form and thiazolidinediones in immediate-release form are commercially available in the form of tablets under the brand name Actoplus Met XR comprising an osmotic core of metformin and pioglitazone coating over the core. U.S. Pat. No. 6,099,859 covers the marketed formulation.

WO 2004/026241, WO 99/47125, U.S. patent application Ser. Nos. 2005/0249809 and 2004/0161462 and U.S. Pat. No. 6,099,862 disclose a metformin extended-release tablet coated with an immediate-release coating containing an antihyperglycemic or a hypoglycemic drug. The metformin cores are present as osmotic cores.

WO 2004/069229 discloses a multilayered formulation comprising extended-release biguanide and immediate-release thiazolidinedione.

There is a need in the art to develop drug formulations in which biguanide is present in extended-release form and thiazolidinedione is present in an immediate-release form that are simpler and more cost effective.

### SUMMARY OF THE INVENTION

In one general aspect, the present invention provides for a pharmaceutical composition that includes a biguanide in an extended-release form and a thiazolidinedione in an immediate-release form.
In another general aspect, the present invention provides for a pharmaceutical composition of a biguanide and a thiazolidinedione that includes:
   (i) a biguanide core which includes a therapeutically effective amount of biguanide or its pharmaceutically effective salts and one or more pharmaceutically acceptable excipients;
   (ii) optionally a seal coat;
   (iii) an extended-release coat which includes one or more rate-controlling materials wherein there is no passageway in the coat;
   (iv) a thiazolidinedione coat which includes a therapeutically effective amount of a thiazolidinedione or its pharmaceutically effective salts and one or more pharmaceutically acceptable excipients; and
   (v) optionally a film coat.

In another general aspect, the present invention provides for a pharmaceutical composition of metformin and pioglitazone that includes:
(i) a metformin core which includes a therapeutically effective amount of metformin or its pharmaceutically effective salts and one or more pharmaceutically acceptable excipients;
(ii) optionally a seal coat;
(iii) an extended-release coat which includes one or more rate-controlling materials, wherein there is no passageway in the coat;
(iv) a pioglitazone coat which includes a therapeutically effective amount of pioglitazone or its pharmaceutically effective salt; and one or more pharmaceutically acceptable excipients; and
(v) optionally a film coat.

Embodiments of the composition may include one or more of the following features. For example, the composition may be in the form of tablets and capsules. The capsules may include one or more of aggregated particles, pellets, mini tablets, tablets, beads or granules.

In one embodiment, the biguanide may be layered onto a pharmaceutically inert core or seed. The inert core or seed may be hydrosoluble or hydroinsoluble.

The biguanide core of the present invention may include one or more pharmaceutically acceptable excipients selected from one or more of fillers, binders, disintegrants, anti adherents, lubricants, glidants, osmogents, coloring agents and flavoring agents.

The biguanide core may additionally contain one or more absorption enhancers.

The seal coat may be applied over the biguanide core or over the extended-release coat.

The rate-controlling materials used in the composition include hydrophilic polymers, hydrophobic polymers, water-swellable polymers, other hydrophobic materials, and mixtures thereof.

The biguanide used in the composition may include one or more of metformin, phenformin, buformin and their pharmaceutically acceptable salts, solvates, polymorphs, enantiomers, isomers, or mixtures thereof. For example, the biguanide is metformin or its pharmaceutically acceptable salts, solvates, polymorphs, enantiomers, isomers, or mixtures thereof.

The thiazolidinedione used in the composition includes pioglitazone, rosiglitazone, troglitazone, ciglitazone, englitazone, and their pharmaceutically acceptable salts, solvates, polymorphs, enantiomers, isomers, or mixtures thereof. For example, the thiazolidinedione is pioglitazone or its pharmaceutically acceptable salts, solvates, polymorphs, enantiomers, isomers, or mixtures thereof.

The biguanide layer includes a mixture of biguanide and thiazolidinedione or the thiazolidinedione layer includes a mixture of thiazolidinedione and biguanide or both the biguanide and thiazolidinedione layers include a mixture of biguanide and thiazolidinedione.

The pharmaceutical composition of the present invention further includes an additional antidiabetic agent.

In another general aspect, the present invention provides for a process for preparing a pharmaceutical composition. The process includes the steps of:
(i) preparing the cores which include a biguanide or its pharmaceutically effective salts and one or more pharmaceutically acceptable excipients;
(ii) optionally coating the cores with a seal coat;
(iii) coating the biguanide cores of step(ii) with an extended-release coating composition which includes one or more rate-controlling materials;
(iv) coating the extended-release core with a thiazolidinedione coating composition which includes thiazolidinedione or its pharmaceutically effective salts and one or more pharmaceutically acceptable excipients; and
(v) optionally providing a film coat.

In another general aspect, the present invention provides for a method for treating diabetes by administering to a person in need thereof a pharmaceutical composition of a biguanide and a thiazolidinedione. The composition includes:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials, wherein there is no passageway in the coat;
(iv) a thiazolidinedione coat which includes a therapeutically effective amount of a thiazolidinedione or its pharmaceutically effective salts; and one or more pharmaceutically acceptable excipients; and
(v) optionally a film coat.

The details of one or more embodiments of the inventions are set forth in the description below. Other features and objects of the invention will be apparent from the description and examples.

### DESCRIPTION OF THE INVENTION

The present invention provides for a pharmaceutical composition that includes a biguanide in extended-release form and a thiazolidinedione in an immediate-release form.

The present invention also provides for a pharmaceutical composition of a biguanide and a thiazolidinedione that includes:
(i) a biguanide core which includes a therapeutically effective amount of biguanide or its pharmaceutically effective salts and one or more pharmaceutically acceptable excipients;
(ii) optionally a seal coat;
(iii) an extended-release coat which includes one or more rate-controlling materials, wherein there is no passageway in the coat;
(iv) a thiazolidinedione coat which includes a therapeutically effective amount of a thiazolidinedione or its pharmaceutically effective salts and one or more pharmaceutically acceptable excipients; and
(v) optionally a film coat.

The biguanide used in the composition may include one or more of metformin, phenformin, buformin and their pharmaceutically acceptable salts, solvates, polymorphs, enantiomers, isomers, or mixtures thereof. For example, the biguanide may be metformin or its pharmaceutically acceptable salts, solvates, polymorphs, enantiomers, isomers, or mixtures thereof. The amount of biguanide in the present composition may range from 25 mg to 2000 mg.

The extended-release biguanide core may be present as aggregated particles, pellets, mini tablets, tablets, beads or granules. Alternatively, the biguanide may be layered onto a pharmaceutically inert core or seed. The inert core or seed may be hydrosoluble or hydroinsoluble.

The hydrosoluble inert cores may be one or more of soluble cores, such as sugar spheres having sugars like dextrose, lactose, anhydrous lactose, spray-dried lactose, lactose monohydrate, mannitol, starches, sorbitol, sucrose, or mixtures thereof.

The hydroinsoluble cores may include one or more of glass particles/beads or silicon dioxide, calcium phosphate dihydrate, dicalcium phosphate, calcium sulfate dihydrate, microcrystalline cellulose (e.g., Avicel®), silicified microcrystalline cellulose (e.g., Prosolv®), cellulose derivatives, powdered cellulose (e.g., Elcema™ G 250 manufactured by Degussa), or mixtures thereof.

The biguanide core of the present invention includes one or more pharmaceutically acceptable excipients. The pharmaceutically acceptable excipients are those known to those skilled in the art and may be selected from one or more of fillers, diluents, binders, disintegrants, anti adherents, lubricants, glidants, osmogents, coloring agents and flavoring agents.

Examples of fillers or diluents include calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose. Further examples of the diluents include sodium chloride, hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, or mixtures thereof.

Examples of binders include povidones, starches, corn starch, pregelatinized starch, microcrystalline celluloses (MCC), silicified MCC (e.g., Prosolv® HD 90), microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcelluloses or hypromelloses (e.g., Klucel®EF, Methocel®E5 premium), or mixtures thereof.

Examples of disintegrants include starch, croscarmellose sodium, crospovidone, sodium starch glycolate, or mixtures thereof.

Examples of anti adherents include magnesium stearate, talc, calcium stearate, glyceryl behenate, polyethylene glycols, hydrogenated vegetable oil, mineral oil, stearic acid, and combinations of the foregoing materials.

Examples of lubricants and glidants include colloidal anhydrous silica, stearic acid, magnesium stearate, calcium stearate, talc, hydrogenated castor oil, sucrose esters of fatty acids, microcrystalline wax, yellow beeswax, white beeswax, sodium benzoate, sodium acetate, sodium chloride, or mixtures thereof.

Examples of osmogents that may be used as swelling agent in the upper compressed layer, include sodium or potassium chloride, sodium or potassium hydrogen phosphate, sodium or potassium dihydrogen phosphate, salts of organic acids, such as sodium or potassium acetate, magnesium succinate, sodium benzoate, sodium citrate or sodium ascorbate; carbohydrates, such as mannitol, sorbitol, arabinose, ribose, xylose, glucose, fructose, mannose, galactose, sucrose, maltose, lactose, raffinose; water-soluble amino acids, such as glycine, leucine, alanine, or methionine; urea, and the like; a polymer consisting of acrylic acid lightly cross-linked with polyallylsucrose mixtures thereof. The drugs such as biguanides may itself act as an osmogent.

The coloring agents and flavoring agents of the present invention may be selected from any FDA approved colors and flavors for oral use.

The biguanide core may additionally contain one or more absorption enhancers. The absorption enhancers include surfactants (anionic, cationic, amphoteric), bile salts, calcium chelating agents, fatty acids, cyclodextrins, chitosan, or mixtures thereof. Particularly, the absorption enhancers include surfactants.

The biguanide core may be prepared by any pharmaceutically acceptable technique that achieves uniform blending, e.g., dry blending, dry granulation, wet granulation, compaction, and fluidized bed granulation. For example, the core formulation of the present invention is fabricated by compression into tablet.

The rate-controlling materials used in the extended-release coat composition include hydrophilic polymers, hydrophobic polymers, water swellable polymers, other hydrophobic materials, or mixtures thereof. The extended-release coat is permeable to the passage of water, biological fluids and the drugs in the core. Further, the extended-release coat does not contain any passageway.

Examples of hydrophilic polymers include one or more of cellulose derivatives, such as, hydroxypropylmethylcellulose, hydroxypropylcellulose, hydroxyethylcellulose, hydroxymethylcellulose, methylcellulose, carboxymethylcellulose, sodium carboxymethylcellulose, carboxymethylcellulose calcium, or combinations thereof; ammonium alginate, sodium alginate, potassium alginate, calcium alginate, propylene glycol alginate, alginic acid, polyvinyl alcohol, povidone, carbomer, xanthan gum, guar gum, locust bean gum, potassium pectate, potassium pectinate, polyvinylpyrrolidone, polysaccharide, polyalkylene oxides, polyalkyleneglycol, starch and derivatives; and mixtures thereof.

Examples of hydrophobic polymers include ethyl cellulose, hydroxyethylcellulose, cellulose acetate, cellulose acetate butyrate, cellulose acetate phthalate, cellulose acetate trimellitate, hydroxypropyl methylcellulose phthalate, poly (alkyl) methacrylate, and copolymers of acrylic or methacrylic acid esters, ammonio methyacrylate copolymer (Eudragit® RL or Eudragit® RS), methyacrylic acid copolymers (Eudragit® L or Eudragit® S), methacrylic acid-acrylic acid ethyl ester copolymer (Eudragit® L 100-5), methacrylic acid esters neutral copolymer (Eudragit® NE 30D™), dimethylaminoethylmethacrylate-methacrylic acid esters copolymer (Eudragit® E 100™), vinyl methyl ether/maleic anhydride copolymers, their salts and esters (Gantrez®), polyvinyl acetate and mixture thereof.

Examples of water-swellable polymers include polyethylene oxide having a molecular weight of 100,000 to 8,000,000; poly(hydroxy alkyl methacrylate) having a molecular weight of from 30,000 to 5,000,000; poly(vinyl) alcohol, having a low acetal residue, which is cross-linked with glyoxal, formaldehyde or glutaraldehyde and having a degree of polymerization of from 200 to 30,000; a mixture of methyl cellulose, cross-linked agar and carboxymethyl cellulose; a water insoluble, water-swellable copolymer produced by forming a dispersion of a finely divided copolymer of maleic anhydride with styrene, ethylene, propylene, butylene or isobutylene cross-linked with from 0.001 to 0.5 moles of saturated cross-linking agent per mole of maleic anhydride in the copolymer; Carbopol® carbomer which is as acidic carboxy polymer having a molecular weight of 450,000 to 4,000,000; Cyanamer® polyacrylamides; cross-linked water-swellable indene-maleic anhydride polymers; Goodrich® polyacrylic acid having a molecular weight of 80,000 to 200,000; starch graft copolymers; Aqua-Keeps® acrylate polymer polysaccharides composed of condensed glucose units such as diester cross-linked polyglucan and the like; Amberlite® ion exchange resins; Explotab® sodium starch glycolate; Ac-Di-Sol® croscarmellose sodium; and combinations of the foregoing materials.

Examples of other hydrophobic materials include waxes such as beeswax, carnauba wax, microcrystalline wax, candelilla wax, spermaceti, montan wax, hydrogenated vegetable oil, lecithin, hydrogenated cottonseed oil, hydrogenated tallow, paraffin wax, shellac wax, petrolatum, ozokerite and the like as well as synthetic waxes, e.g., polyethylene, and the like; fatty acids, such as stearic acid, palmitic acid, lauric acid, eleostearic acids, and the like; fatty alcohols, such as lauryl alcohol, cetostearyl alcohol, stearyl alcohol, cetyl alcohol and myristyl alcohol; fatty acid esters, such as glyceryl monostearate, glycerol monooleate, acetylated monoglycerides, tristearin, tripalmitin, cetyl esters wax, glyceryl palmitostearate and glyceryl behenate; vegetable oil, such as hydrogenated castor oil; mineral oil; and combinations of the foregoing materials.

The extended-release coat composition may additionally include plasticizers selected from propylene glycol, triethylene glycol, oleic acid, ethyleneglycol monoleate, triethyl citrate, triacetin, diethyl phthalate, glyceryl monostearate, dibutyl sebacate, acetyl triethyl citrate, acetyl tributyl citrate, castor oil or mixtures thereof; opacifiers selected from titanium dioxide, talc, calcium carbonate, behenic acid, cetyl alcohol or mixtures thereof.

The coating compositions may be coated on the biguanide core by conventional methods such as drug layering, dry compression, deposition and printing.

The coating composition may be applied as a solution or dispersion of rate-controlling materials. Examples of solvents used for preparing a solution/dispersion of rate-controlling materials include methylene chloride, isopropyl alcohol, acetone, methanol, ethanol, water, or mixtures thereof.

A seal coating composition may be coated over the biguanide core or over the extended-release coat. The polymers used to provide seal coating may include one or more of a hydrophilic polymer. Examples include hydroxypropylcellulose, hydroxypropylisopropylcellulose, methoxypropyl cellulose, hydroxypropylmethylcellulose, hydroxypropylpentylcellulose, hydroxypropylhexylcellulose, or mixtures thereof. The seal coating may optionally be opacified.

The thiazolidinedione coat includes a therapeutically effective amount of a thiazolidinedione or its pharmaceutically effective salts and one or more pharmaceutically acceptable excipients known to those skilled in the art, and may be selected from one or more of wicking agents, agents, plasticizers, opacifiers and colorants.

The thiazolidinedione used in the composition includes pioglitazone, rosiglitazone, troglitazone, ciglitazone, englitazone, and their pharmaceutically acceptable salts, solvates, polymorphs, enantiomers, isomers, or mixtures thereof. For example, the thiazolidinedione is pioglitazone or its pharmaceutically acceptable salts, solvates, polymorphs, enantiomers, isomers, or mixtures thereof. The amount of thiazolidinedione in the present composition may range from 15 mg to 60 mg.

A wicking agent is defined as any material with the ability to draw water into the porous network of a delivery device. Examples of wicking agents include silicon dioxide (Syloid® 244FP by Grace Davison Discovery Sciences), kaolin, titanium dioxide, alumina, niacinamide, sodium lauryl sulfate, low molecular weight polyvinyl pyrrolidone, m-pyrol, bentonite, magnesium aluminum silicate, polyester, and polyethylene. Non-swellable wicking agents, examples of which include sodium lauryl sulfate, colloidal silicon dioxide, and low molecular weight polyvinylpyrrolidone, are preferred.

Examples of wetting agents include hydrophilic surfactants, hydrophobic surfactants, or mixtures thereof. The hydrophilic surfactants may be selected from one or more of non-ionic surfactants, ionic surfactants or mixtures thereof.

Examples of hydrophobic surfactants include one or more of alcohols; polyoxyethylene alkylethers; fatty acids; glycerol fatty acid monoesters; glycerol fatty acid diesters; acetylated glycerol fatty acid monoesters; acetylated glycerol fatty acid diesters, lower alcohol fatty acid esters; polyethylene glycol fatty acid esters; polyethylene glycol glycerol fatty acid esters; polypropylene glycol fatty acid esters; polyoxyethylene glycerides; lactic acid derivatives of monoglycerides; lactic acid derivatives of diglycerides; propylene glycol diglycerides; sorbitan fatty acid esters; polyoxyethylene sorbitan fatty acid esters; polyoxyethylene-polyoxypropylene block copolymers; polyethyleneglycols as esters or ethers, polyethoxylated castor oil; polyethoxylated hydrogenated castor oil, polyethoxylated fatty acid from castor oil or polyethoxylated fatty acid from castor oil or polyethoxylated fatty acid from hydrogenated castor oil.

Examples of non-ionic surfactants include alkylglucosides; alkylmaltosides; alkylthioglucosides; lauryl macrogolglycerides; caprylocaproyl macrogolglycerides, polyoxyethylene alkyl ethers; polyoxyethylene alkylphenols; polyethylene glycol fatty acid esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction products of polyols and at least one member of the group consisting of fatty acids, glycerides, vegetable oils, hydrogenated vegetable oils, and sterols; sugar esters, sugar ethers; sucroglycerides; and mixtures thereof.

Examples of ionic surfactants include alkyl ammonium salts; bile acids and salts, analogues, and derivatives thereof; fatty acid derivatives of amino acids, oligopeptides, and polypeptides; glyceride derivatives of amino acids, oligopeptides, and polypeptides; acyl lactylates; monoacetylated tartaric acid esters of monoglycerides, monoacetylated tartaric acid esters of diglycerides, diacetylated tartaric acid esters of monoglycerides, diacetylated tartaric acid esters of diglycerides; succinylated monoglycerides; citric acid esters of monoglycerides; citric acid esters of diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate, and mixtures thereof.

The examples of plasticizers include propylene glycol, triethylene glycol, oleic acid, ethyleneglycol monoleate, triethyl citrate, triacetin, diethyl phthalate, glyceryl monostearate, dibutyl sebacate, acetyl triethyl citrate, acetyl tributyl citrate, castor oil, or mixtures thereof.

The examples of opacifiers include titanium dioxide, talc, calcium carbonate, behenic acid, cetyl alcohol or mixtures thereof.

The immediate-release thiazolidinedione layer may further include one or more film forming polymers. The film-forming polymers may be hydrophilic polymers.

The thiazolidinedione coating composition may be applied as solution/dispersion of thiazolidinedione over the extended-release coat. Example of solvents used for preparing a solution/dispersion of thiazolidinedione include methylene chloride, isopropyl alcohol, acetone, methanol, ethanol, water or mixtures thereof.

The pharmaceutical composition may optionally be coated with one or more layers of a film coat comprising film forming agents and/or pharmaceutically acceptable excipients.

Example of film forming agents include ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate waxes, such as, polyethylene glycol; methacrylic acid polymers, such as, Eudragit® RL and RS; or mixture thereof. Alternatively, commercially available coating compositions comprising film-forming polymers marketed under various trade names, such as Opadry® may also be used for coating.

The film forming agents may be applied as a solution/dispersion of coating ingredients. Examples of solvents used for preparing a solution/dispersion of the coating ingredients include methylene chloride, isopropyl alcohol, acetone, methanol, ethanol, water or mixtures thereof.

The various coats of the composition may be coated using a conventional coating pan, a spray coater, a rotating perforated pan, or an automated system, such as a centrifugal fluidizing (CF) granulator, a fluidized bed process, or any other suitably automated coating equipment.

The present invention is illustrated below by reference to the following examples. However, one skilled in the art will appreciate that the specific methods and results discussed are merely illustrative of the invention, and not to be construed as limiting the invention.

### EXAMPLE 1

| | |
|---|---|
| Ingredients | Percent w/w of tablet |

Core Composition

| | |
|---|---|
| Metformin Hydrochloride | 68.49 |
| Microcrystalline Cellulose | 13.70 |
| Polyvinylpyrrolidone | 4.11 |
| Hydrophobic Silicon Dioxide | 0.43 |
| Magnesium Stearate | 0.43 |

ER Coating (10-15%)

| | |
|---|---|
| Ethyl Cellulose | 8.20 |
| Hydroxypropyl Methylcellulose | 0.43 |
| Talc | 0.43 |
| Isopropyl Alcohol | qs |
| Purified Water | qs |

Active Coat

| | |
|---|---|
| Pioglitazone Hydrochloride | 2.05 |
| Equivalent To Pioglitazone | |
| Polysorbate-80 | 0.03 |
| Hydroxypropyl Methylcellulose E-5 | 0.25 |
| Isopropyl Alcohol | qs |
| Purified Water | qs |

Film Coating

| | |
|---|---|
| Opadry ® Oys White | 1.44 |
| Purified Water | qs |

### Procedure

### Core Tablets

1. Metformin hydrochloride and microcrystalline cellulose were mixed uniformly and granulated with polyvinylpyrrolidone solution in water.

2. The granules were dried in the fluidized bed dryer and the dried granules were mixed with hydrophobic colloidal silicon dioxide and magnesium stearate.

3. The blend of step 2 was compressed into core tablets. ER Coating:

4. Ethyl Cellulose, hydroxypropyl methylcellulose and talc were dispersed in isopropyl alcohol-water mixture.

5. The tablet of step 3 were coated with the step 4 dispersion.

### Active Coat

6. The isopropyl alcohol-water mixture was taken in a container and hydroxypropyl methylcellulose E-5 was added to it followed by Polysorbate-80 and Pioglitazone hydrochloride.

7. The ER coated tablets were coated with the dispersion of step 6 in a pan coater.

Film Coating 8. The tablets of step 7 were coated with Opadry coating.

### EXAMPLE 2

| | |
|---|---|
| Ingredients | Percent w/w of tablet |

Core Composition

| | |
|---|---|
| Metformin Hydrochloride | 64.52 |
| Sodium Carboxymethyl Cellulose | 3.23 |
| Microcrystalline Cellulose | 6.45 |
| Hydroxypropyl Methylcellulose | 11.29 |
| Magnesium Stearate | 0.65 |
| Silicon Dioxide | 0.65 |
| Hydroxypropyl Methylcellulose E-5 | 1.61 |
| Purified Water | qs |

ER Coating (0-15%)

| | |
|---|---|
| Ethyl Cellulose | 7.34 |
| Hydroxypropyl Methylcellulose | 0.41 |
| Talc | 0.39 |
| Isopropyl Alcohol | qs |
| Purified Water | qs |

Coat Composition

| | |
|---|---|
| Pioglitazone Hydrochloride | 1.94 |
| Equivalent To Pioglitazone | |
| Polysorbate-80 | 0.02 |
| Hydroxypropyl Methylcellulose E-5 | 0.24 |
| Isopropyl Alcohol | qs |
| Purified Water | qs |

Film Coating

| | |
|---|---|
| Opadry ® Oys White | 1.29 |
| Purified Water | qs |

### Procedure

### Core Tablets

1. Metformin hydrochloride, sodium carboxymethyl cellulose, microcrystalline cellulose and hydroxypropyl methylcellulose were mixed uniformly and granulated with Methocel E-5.

2. The granules were dried in the fluidized bed dryer and lubricated with magnesium stearate and silicon dioxide.

3. The blend of step 2 was compressed into core tablets.

### ER Coating

4. Ethyl Cellulose, hydroxypropyl methylcellulose and talc were dispersed in isopropyl alcohol-water mixture.

5. The tablets of step 3 were coated with the step 4 dispersion.

### Pioglitazone Coating

6. The isopropyl alcohol-water mixture was taken in a container and hydroxypropyl methylcellulose E-5 was added to it followed by Polysorbate-80 and Pioglitazone hydrochloride.

7. The ER coated tablets were coated with the dispersion of step 6 in a pan coater.

### Film Coating

8. The tablets of step 7 were coated with Opadry® coating.

### EXAMPLE 3

| | |
|---|---|
| Ingredients | Percent w/w of tablet |

Core Composition

| | |
|---|---|
| Metformin Hydrochloride | 68.49 |
| Polyvinylpyrrolidone | 4.79 |
| Sodium Lauryl Sulphate | 1.71 |
| Sodium Chloride | 1.71 |
| Magnesium Stearate | 0.68 |
| Purified Water | qs |

ER Coating (5%-20%)

| | |
|---|---|
| Ethyl Cellulose | 13.23 |
| Hydroxypropyl Methylcellulose | 1.47 |
| Diethyl Phthalate | 1.47 |
| Talc | 0.75 |
| Isopropyl Alcohol | qs |
| Purified Water | qs |

Pioglitazone Layer

| | |
|---|---|
| Pioglitazone Hydrochloride | 2.05 |
| Equivalent To Pioglitazone | |
| Hydroxypropyl Methylcellulose | 2.05 |
| Polysorbate 80 | 0.03 |
| Talc | 1.53 |
| Isopropyl Alcohol | qs |
| Purified Water | qs |

### Procedure

### Core Tablets

1. Metformin hydrochloride, sodium lauryl sulphate and sodium chloride were mixed uniformly and granulated with polyvinylpyrrolidone in rapid mixer granulator.

2. The granules were dried in the fluidized bed dryer and lubricated with magnesium stearate.

3. The blend of step 2 was compressed into core tablets.

### ER Coating

4. Ethyl Cellulose, hydroxypropyl methylcellulose, diethyl phthalate and talc were dispersed in an isopropyl alcohol-water mixture.

5. The tablets of step 3 were coated with the step 4 dispersion.

### Pioglitazone Layer

6. Pioglitazone hydrochloride, hydroxypropyl methylcellulose, polysorbate 80 and talc were dispersed in the isopropyl alcohol-water mixture.

7. The ER coated tablets were coated with the dispersion of step 6.

### EXAMPLE 4

| | |
|---|---|
| Ingredients | Percent w/w of tablet |

Core Composition

| | |
|---|---|
| Metformin Hydrochloride | 71.79 |
| Microcrystalline Cellulose | 3.59 |
| Sodium Lauryl Sulphate | 3.59 |
| Polyvinylpyrrolidone | 5.03 |
| Magnesium Stearate | 0.72 |
| Isopropyl Alcohol | qs |

Extended Release Coating

| | |
|---|---|
| Cellulose Acetate | 8.69 |
| Triacetin | 0.57 |
| Polyethylene Glycol | 0.57 |
| Hydroxypropyl Methylcellulose | 2.87 |

Pioglitazone Layer

| | |
|---|---|
| Pioglitazone Hydrochloride | 2.15 |
| Equivalent To Pioglitazone | |
| Sodium Lauryl Sulphate | 0.14 |
| Hydroxypropyl Methylcellulose | 0.29 |
| Isopropyl Alcohol | qs |
| Purified Water | qs |

### Procedure

### Core Tablets

1. Metformin hydrochloride, microcrystalline cellulose and sodium lauryl sulphate were mixed uniformly and granulated with polyvinylpyrrolidone solution in isopropyl alcohol.

2. The granules were dried in the fluidized bed dryer and lubricated with magnesium stearate.

3. The blend of step 2 was compressed into core tablets.

### ER Coating

4. Cellulose acetate was dissolved in acetone followed by addition of triacetin, polyethylene glycol, water and hydroxypropyl methylcellulose under stirring.

5. The tablets of step 3 were coated with the step 4 dispersion.

### Pioglitazone Layer

6. Hydroxypropyl methylcellulose and sodium lauryl sulphate were dissolved in water followed by the addition of isopropyl alcohol.

7. Pioglitazone hydrochloride was dispersed in the solution of step 6.

8. The ER coated tablets were coated with the dispersion of step 7.

### EXAMPLE 5

| | |
|---|---|
| | Percent w/w |
| Ingredients | of tablet |

Core Composition

| | |
|---|---|
| Metformin Hydrochloride | 85-60 |
| Microcrystalline Cellulose | 1-5.0 |
| Sodium Lauryl Sulphate | 0.5-3.0 |
| Polyvinylpyrrolidone | 4.0-7.5 |
| Magnesium Stearate | 0.2-0.60 |
| Purified Water/Isopropyl Alcohol | Qs |

Seal Coating (Optional)

| | |
|---|---|
| Hydroxypropyl Methylcellulose | 1.0-3.0 |
| Isopropyl Alcohol | Q.S |

Extended Release Coating (3%-15% Weight Buildup)

| | |
|---|---|
| Cellulose Acetate | 2.5-7.5 |
| Triacetin | 0.1-0.5 |
| Polyethylene Glycol 400 | 0.2-0.60 |
| Polyethylene Glycol 6000 or Hydroxypropyl | 0.2-2.5 |
| Methylcellulose | |
| Acetone | Qs |
| Purified Water | Qs |

Pioglitazone Layer

| | |
|---|---|
| Pioglitazone Hydrochloride Equivalent to Pioglitazone | 3.0-1.5 |
| Lactose Monohydrate | 0.05-5.00 |
| Sodium Lauryl Sulphate (Optional) | 0.00-1.5 |
| Hydroxypropylcellulose | 0.2-5.0 |
| Purified Water/Non-Aqueous Solvent | Qs |

### Procedure

### Core Tablets

1. Metformin hydrochloride, microcrystalline cellulose and sodium lauryl sulphate were mixed uniformly and granulated with polyvinylpyrrolidone solution in isopropyl alcohol.

2. The granules were dried in the fluidized bed dryer and lubricated with magnesium stearate.

3. The blend of step 2 was compressed into core tablets.

### Seal Coating

4. Hydroxypropyl methylcellulose was dissolved in isopropyl alcohol.

5. The tablets of step 3 were coated with the step 4 dispersion.

### ER Coating

6. Cellulose acetate was dissolved in acetone followed by addition of triacetin, polyethylene glycol, water and hydroxypropyl methylcellulose under stirring.

7. The seal coated tablets of step 5 were coated with the step 6 dispersion.

### Pioglitazone Layer

8. Lactose, hydroxypropylcellulose and sodium lauryl sulphate were dissolved in water/non-aqueous solvent.

9. Pioglitazone hydrochloride was dispersed in the solution of step 8.

10. The ER coated tablets were coated with the dispersion of step 9.

### EXAMPLE 6

| | |
|---|---|
| Ingredients | Percent w/w of Tablet |

Core Composition

| | |
|---|---|
| Metformin HCl | 75.97 |
| Microcrystalline Cellulose | 3.80 |
| Polyvinylpyrrolidone | 7.60 |
| Sodium Lauryl Sulphate | 0.76 |
| Magnesium Stearate | 0.76 |
| Isopropyl Alcohol | q.s. |

Seal Coating (2.5% Weight Buildup; Range 2%-3%)

| | |
|---|---|
| Hydroxypropyl Methylcellulose | 2.22 |
| Isopropyl Alcohol (85 parts) | q.s. |
| Water (15 Parts) | q.s. |

ER Coating: 2% Weight Buildup (1%-3% Buildup)

| | |
|---|---|
| Cellulose Acetate | 1.55 |
| Triacetin | 0.09 |
| Polyethylene Glycol 400 | 0.18 |
| Acetone | q.s. |

Pioglitazone Coating

| | |
|---|---|
| Pioglitazone Hcl Equiv To Pioglitazone | 2.51 |
| Lactose Monohydrate | 2.28 |
| Hydroxypropylcellulose | 2.28 |
| Purified Water | q.s. |

### Procedure

### Core Tablets

1. Metformin hydrochloride, microcrystalline cellulose and sodium lauryl sulphate were mixed uniformly and granulated with polyvinylpyrrolidone solution in isopropyl alcohol.

2. The granules were dried in the fluidized bed dryer and lubricated with magnesium stearate.

3. The blend of step 2 was compressed into core tablets.

### Seal Coating

4. Hydroxypropyl methylcellulose was dissolved in isopropyl alcohol.

5. The tablets of step 3 were coated with the step 4 dispersion.

### ER Coating

6. Cellulose acetate was dissolved in acetone followed by addition of triacetin and polyethylene glycol under stirring.

7. The seal coated tablets of step 5 were coated with the step 6 dispersion.

### Pioglitazone Layer

8. Lactose and hydroxypropylcellulose were dissolved in water.

9. Pioglitazone hydrochloride was dispersed in the solution of step 8.

10. The ER coated tablets were coated with the dispersion of step 9.

### EXAMPLE 7

| | |
|---|---|
| Ingredients | Percent w/w of Tablet |

Core Composition

| | |
|---|---|
| Metformin HCl | 70.22 |
| Microcrystalline Cellulose | 3.51 |
| Polyvinylpyrrolidone | 7.02 |
| Sodium Lauryl Sulphate | 0.70 |
| Magnesium Stearate | 0.70 |
| Isopropyl Alcohol | Q.S. |

Seal Coating (2.5% Weight Buildup; Range 2%-3%)

| | |
|---|---|
| Hydroxypropyl Methylcellulose | 2.05 |
| Isopropyl Alcohol (85parts) | Q.S. |
| Water (15 Parts) | Q.S. |

ER COATING: (8%-10% Weight Buildup)

| | |
|---|---|
| Cellulose Acetate | 4.38 |
| Polyethylene Glycol 6000 | 1.01 |
| Triacetin | 0.67 |
| Polyethylene Glycol 400 | 0.67 |
| Acetone | Q.S. |

Pioglitazone Coating

| | |
|---|---|
| Pioglitazone HCl Equiv To Pioglitazone | 2.32 |
| Lactose Monohydrate | 2.87 |
| Hydroxypropylcellulose | 1.76 |
| Silicon Dioxide | 2.11 |
| Purified Water | Q.S. |

### Procedure

### Core Tablets

1. Metformin hydrochloride, microcrystalline cellulose and sodium lauryl sulphate were mixed uniformly and granulated with polyvinylpyrrolidone solution in isopropyl alcohol.

2. The granules were dried in the fluidized bed dryer and lubricated with magnesium stearate.

3. The blend of step 2 was compressed into core tablets.

### Seal Coating

4. Hydroxypropyl methylcellulose was dissolved in isopropyl alcohol.

5. The tablets of step 3 were coated with the step 4 dispersion.

### ER Coating

6. Cellulose acetate was dissolved in acetone followed by addition of triacetin and polyethylene glycol under stirring.

7. The seal coated tablets of step 5 were coated with the step 6 dispersion.

### Pioglitazone Layer

8. Lactose, hydroxypropylcellulose and silicon dioxide were dissolved in water.

9. Pioglitazone hydrochloride was dispersed in the solution of step 8.

10. The ER coated tablets were coated with the dispersion of step 9.

### EXAMPLE 8

| | | |
|---|---|---|
| | Percent w/w | Percent w/w |
| | of Tablet | of Tablet |
| Ingredients | (15/1000 mg) | (30/1000 mg) |

Core Composition

| | | |
|---|---|---|
| Metformin HCl | 72.60 | 71.73 |
| Sodium Lauryl Sulphate | 2.90 | 2.87 |
| Polyvinylpyrrolidone | 7.26 | 7.17 |
| Purified Water | Q.S. | Q.S. |
| Magnesium Stearate | 0.41 | 0.41 |

Seal Coating

| | | |
|---|---|---|
| Hydroxypropyl | 1.66 | 1.64 |
| Methylcellulose | | |
| Isopropyl Alcohol | Q.S. | Q.S. |
| Purified Water | Q.S. | Q.S. |

ER COATING

| | | |
|---|---|---|
| Cellulose Acetate | 4.96 | 4.90 |
| Polyethylene Glycol 6000 | 1.15 | 1.13 |
| Triacetin | 0.76 | 0.75 |
| Polyethylene Glycol 400 | 0.76 | 0.75 |
| Acetone | Q.S. | Q.S. |
| Purified Water | Q.S. | Q.S. |

PIOGLITAZONE COATING

| | | |
|---|---|---|
| Pioglitazone HCl Equiv To | 1.20 | 2.37 |
| Pioglitazone | | |
| Lactose Monohydrate | 2.18 | 2.15 |
| Hydroxypropylcellulose | 2.18 | 2.15 |
| Purified Water | Q.S. | Q.S. |

FILM COATING

| | | |
|---|---|---|
| Opadry ® White | 1.96 | 1.96 |
| Purified Water | Q.S. | Q.S. |

### Procedure

### Core Tablets

1. Metformin hydrochloride and sodium lauryl sulphate were mixed uniformly and granulated with polyvinylpyrrolidone solution in purified water.

2. The granules were dried in the fluidized bed dryer and lubricated with magnesium stearate.

3. The blend of step 2 was compressed into core tablets.

### Seal Coating

4. Hydroxypropyl methylcellulose was dispersed in isopropyl alcohol followed by addition of purified water.

5. The tablets of step 3 were coated with the step 4 dispersion.

### ER Coating

6. Cellulose acetate was dissolved in acetone followed by addition of triacetin and polyethylene glycol under stirring.

7, Purified water was added slowly under stirring to the step 6 dispersion.

8. The seal coated tablets of step 5 were coated with the step 7 dispersion.

### Pioglitazone Layer

9. Lactose and hydroxypropylcellulose were dissolved in purified water.

10. Pioglitazone hydrochloride was dispersed in the solution of step 9.

11. The ER coated tablets were coated with the dispersion of step 10.

### Film Coating

12. Film coating solution was prepared by dispersing Opadry® white in purified water.

13. The pioglitazone coated tablets were coated with the solution of step 12.

While several particular forms of the invention have been illustrated and described, it will be apparent that various modifications and combinations of the invention detailed in the text can be made without departing from the spirit and scope of the invention.

We claim:

1. A pharmaceutical composition of a biguanide and a thiazolidinedione comprising:
(i) a biguanide core comprising a therapeutically effective amount of a biguanide or its pharmaceutically effective salts and one or more pharmaceutically acceptable excipients;
(ii) optionally a seal coat;
(iii) an extended release coat comprising one or more rate-controlling materials wherein there is no passageway in the coat;
(iv) a thiazolidinedione coat comprising a therapeutically effective amount of a thiazolidinedione or its pharmaceutically effective salts and one or more pharmaceutically acceptable excipients; and
(v) optionally a film coat.

2. The pharmaceutical composition according to claim 1, wherein the biguanide comprises one or more of metformin, phenformin, buformin and their pharmaceutically acceptable salts, solvates, polymorphs, enantiomers, isomers, or mixtures thereof.

3. The pharmaceutical composition according to claim 1, wherein the thiazolidinedione comprises one or more of pioglitazone, rosiglitazone, troglitazone, ciglitazone, englitazone, and their pharmaceutically acceptable salts, solvates, polymorphs, enantiomers, isomers, or mixtures thereof.

4. The pharmaceutical composition according to claim 1, wherein the biguanide is metformin and the thiazolidinedione is pioglitazone.

5. The pharmaceutical composition according to claim 1, wherein the biguanide is layered onto a pharmaceutically inert core or seed.

6. The pharmaceutical composition according to claim 5, wherein the inert core or seed is hydrosoluble or hydroinsoluble.

7. The pharmaceutical composition according to claim 1, wherein the biguanide core comprises one or more pharmaceutically acceptable excipients selected from one or more of fillers, binders, disintegrants, anti adherents, lubricants, glidants, osmogents, coloring agents and flavoring agents.

8. The pharmaceutical composition according to claim 1, wherein the biguanide core additionally contains one or more absorption enhancers.

9. The pharmaceutical composition according to claim 1, wherein the seal coat is applied over the biguanide core or over the extended release coat.

10. The pharmaceutical composition according to claim 1, wherein the rate-controlling material comprises hydrophilic polymers, hydrophobic polymers, water swellable polymers, other hydrophobic materials and mixtures thereof.

11. The pharmaceutical composition according to claim 1, wherein the biguanide layer comprises a mixture of biguanide and thiazolidinedione or the thiazolidinedione layer comprises a mixture of thiazolidinedione and biguanide or both the biguanide and thiazolidinedione layers comprise a mixture of biguanide and thiazolidinedione.

12. The pharmaceutical composition according to claim 1, wherein the composition further comprises an additional antidiabetic agent.

13. A process for preparing a pharmaceutical composition comprising the steps of:
(i) preparing the cores comprising a biguanide or its pharmaceutically effective salts and one or more pharmaceutically acceptable excipients;
(ii) optionally coating the cores with a seal coat;
(iii) coating the biguanide cores of step(ii) with an extended release coating composition comprising one or more rate-controlling materials;
(iv) coating the extended release core with a thiazolidinedione coating composition comprising a thiazolidinedione or its pharmaceutically effective salts and one or more pharmaceutically acceptable excipients; and
(v) optionally providing a film coat.

14. A method for treating diabetes by administering to a person in need thereof a pharmaceutical composition of a biguanide and a thiazolidinedione comprising:
(i) a biguanide core comprising a therapeutically effective amount of biguanide or its pharmaceutically effective salts and one or more pharmaceutically acceptable excipients;
(ii) optionally a seal coat;
(iii) an extended release coat comprising one or more rate-controlling materials, wherein there is no passageway in the coat;
(iv) a thiazolidinedione coat comprising a therapeutically effective amount of a thiazolidinedione or its pharmaceutically effective salts and one or more pharmaceutically acceptable excipients; and
(v) optionally a film coat.
